(19) United States
(12) Patent Application Publication
(10) Pub. No.: US 2013/0029323 A1
(43) Pub. Date: Jan. 31, 2013

(54) SYSTEMS, DEVICES, AND METHODS FOR AUTOMATED CHARACTERIZATION OF A NUCLEIC ACID MOLECULE

(71) Applicant: Adam Michael Briska, Madison, WI (US)

(72) Inventor: Adam Michael Briska, Madison, WI (US)

(73) Assignee: OPGEN, INC., Gaithersburg, MD (US)

(21) Appl. No.: 13/190,942

(22) Filed: Jul. 26, 2011

(51) Int. Cl. C12Q 1/68; C12M 1/40

(52) U.S. Cl. 435/6.1; 435/289.1

(57) ABSTRACT

The invention generally relates to systems, cartridges, and methods for automated characterization of a nucleic acid molecule, in particular, optical mapping of DNA from an organism. In certain embodiments, the invention provides a cartridge for characterizing a nucleic acid molecule, the cartridge including a reaction chamber having a derivatized bottom surface, at least one reagent reservoir, and a pump, in which the reaction chamber, the reagent reservoir, and the pump are fluidically connected to each other.

### FIELD OF THE INVENTION

The invention generally relates to systems, cartridges, and methods for automated characterization of a nucleic acid molecule, in particular, optical mapping of DNA from an organism.

### BACKGROUND

Physical mapping of genomes, e.g., using restriction endonucleases to develop restriction maps, can provide accurate information about the nucleic acid sequences of various organisms. Restriction maps of, e.g., deoxyribonucleic acid (DNA), can be generated by optical mapping. Optical mapping can produce ordered restriction maps by using fluorescence microscopy to visualize restriction endonuclease cutting events on individual labeled DNA molecules.

### SUMMARY

The invention generally relates to systems, cartridges, and methods for automated characterization of a nucleic acid molecule, in particular, optical mapping of DNA from an organism. Systems, devices, and methods of the invention combine capillary flow technology for nucleic acid, e.g., DNA or RNA, deposition onto a surface with computer controlled flow processing. The capillary flow presents the nucleic acid molecules to a derivatized surface in long strands that are captured and held to the surface by electrostatic attraction. Once the nucleic acid molecules have been captured on the surface, reagents (e.g., washing solutions, buffers, enzymes, and nucleic acid stains), are flowed to and from the surface to produce restriction digests. The digests are subsequently imaged, thereby characterizing the nucleic acid molecule. In certain embodiments, the digests are used to construct an optical map of the nucleic acid molecule.

An aspect of the invention provides a cartridge for characterizing a nucleic acid molecule, the cartridge including a reaction chamber having a derivatized bottom surface, at least one reagent reservoir, and a pump, in which the reaction chamber, the reagent reservoir, and the pump are fluidically connected to each other. The cartridge uses microfluidic components to link on-board reagent reservoirs via computer controlled valves and plumbing to a reaction chamber having a derivatized bottom surface. The derivatized bottom surface assists in elongating and fixing nucleic acid molecules, e.g., DNA or RNA, onto a surface so that the nucleic acid molecules remain accessible for enzymatic reactions. In certain embodiments, the derivatized bottom surface is derivatized glass.

The cartridge can be operably linked to a computerized preparation station. Depending on the embodiment, the cartridge can further include at least one of the following: a reagent waste pad, a channel forming device configured to mate with the reaction chamber, a reaction chamber cap, or a heater/cooling device. The heater/cooling device can be located beneath the reaction chamber.

In certain embodiments, the at least one reagent reservoir is a plurality of reservoirs, in which a first reservoir holds a TE wash reagent, a second reservoir holds a buffer, a third reservoir holds an enzyme, and a fourth reservoir holds a nucleic acid stain. Each reservoir can further include a loading port and a computer controlled valve for controlling flow of reagents from the reservoirs to the reaction chamber.

Another aspect of the invention provides a method for characterizing a nucleic acid molecule, the method including applying one or more nucleic acid molecules (e.g., DNA or RNA) to a derivatized bottom surface (e.g., derivatized glass) of a reaction chamber, in which the nucleic acid molecules are elongated and fixed onto the surface so that the nucleic acid molecules remain accessible for enzymatic reactions, flowing reagents to the reaction chamber to wash, enzymatically digest, and stain the nucleic acid molecules to obtain one or more restriction digests of the molecule, and imaging the restriction digests, thereby characterizing the nucleic acid molecule. The method can further include constructing an optical map from the restriction digests.

Prior to the flowing step, the method can further include applying a cap to the reaction chamber. Prior to the flowing step, the method can further include loading the reagents into separate reservoirs, each reservoir being fluidically connected to the reaction chamber. Prior to the flowing step, the method can further include tilting the cartridge at a 60° angle. Flowing can be controlled by pumps and valves that are operably connected to a computerized preparation station.

Another aspect of the invention provides a cartridge for characterizing a nucleic acid molecule, the cartridge including a reaction chamber having a derivatized bottom surface, in which a nucleic acid molecule is elongated and fixed onto the derivatized bottom surface of the reaction chamber so that the nucleic acid molecule remains accessible for enzymatic reactions, at least one reagent reservoir, and a pump, and the reaction chamber, the reagent reservoir, and the pump are fluidically connected to each other.

Another aspect of the invention provides a system for characterizing a nucleic acid molecule, the system including: a cartridge including a reaction chamber having a derivatized bottom surface, in which a nucleic acid molecule is elongated and fixed onto the derivatized bottom surface of the reaction chamber so that the nucleic acid molecule remains accessible for enzymatic reactions, at least one reagent reservoir, and a pump, and the reaction chamber, the reagent reservoir, and the pump are fluidically connected to each other; and a computerized preparation station operably linked to the cartridge, in which the station controls flow of reagents to and from the reaction and chamber.

### DETAILED DESCRIPTION

Optical mapping is a single-molecule technique for production of ordered restriction maps from a single DNA molecule (Samad et al., Genome Res. 5:1-4, 1995). During some applications, individual fluorescently labeled DNA molecules are elongated in a flow of agarose between a coverslip and a microscope slide (in a first-generation method) or fixed onto polylysine-treated glass surfaces (in a second-generation method). Samad et al. supra. The added endonuclease cuts the DNA at specific points, and the fragments are imaged. Id. Restriction maps can be constructed based on the number of fragments resulting from the digest. Id. Generally, the final map is an average of fragment sizes derived from similar molecules. Id.

Optical mapping and related methods are described in U.S. Pat. No. 5,405,519, U.S. Pat. No. 5,599,664, U.S. Pat. No. 6,150,089, U.S. Pat. No. 6,147,198, U.S. Pat. No. 5,720,928, U.S. Pat. No. 6,174,671, U.S. Pat. No. 6,294,136, U.S. Pat. No. 6,340,567, U.S. Pat. No. 6,448,012, U.S. Pat. No. 6,509,158, U.S. Pat. No. 6,610,256, and U.S. Pat. No. 6,713,263. All the cited patents are incorporated by reference herein in their entireties.

Optical Maps are constructed as described in Reslewic et al., Appl Environ Microbiol. 2005 September; 71 (9):5511-22, incorporated by reference herein. Briefly, individual chromosomal fragments from test organisms are immobilized on derivatized glass by virtue of electrostatic interactions between the negatively-charged DNA and the positively-charged surface, digested with one or more restriction endonuclease, stained with an intercalating dye such as YOYO-1 (Invitrogen) and positioned onto an automated fluorescent microscope for image analysis. Since the chromosomal fragments are immobilized, the restriction fragments produced by digestion with the restriction endonuclease remain attached to the glass and can be visualized by fluorescence microscopy, after staining with the intercalating dye. The size of each restriction fragment in a chromosomal DNA molecule is measured using image analysis software and identical restriction fragment patterns in different molecules are used to assemble ordered restriction maps covering the entire chromosome.

Restriction mapping, e.g., optical mapping, can be used in a variety of applications. For example, the methods featured herein can be used to determine a property, e.g., physical and/or chemical property, e.g., size, length, restriction map, weight, mass, sequence, conformational or structural change, pKa change, distribution, viscosity, rates of relaxation of a labeled and/or non-labeled molecule, e.g., an amplicon (e.g., PCR product), of a portion of a genome (e.g., a chromosome), or of an entire genome.

Optical mapping can also be used to identify various organisms, e.g., viruses and prions, and various microorganisms, e.g., bacteria, protists, and fungi, whose genetic information is stored as DNA or RNA by correlating the restriction map of a nucleic acid of an organism with a restriction map database. Such identification methods can be used in diagnosing a disease or disorder. Methods of identifying organisms by restriction mapping are described, e.g., in a U.S. patent application Ser. No. 12/120,586, filed on May 14, 2008, incorporated herein by reference. The methods featured herein can also be used in other diagnostic applications, for example, imaging specific loci or genetic regions for individuals or populations to help identify specific diseases or disorders. Other uses of the methods will be apparent to those skilled in the art.

The invention generally relates to systems, cartridges, and methods for automated characterization of a nucleic acid molecule, in particular, optical mapping of DNA from an organism. An aspect of the invention provides a cartridge for characterizing a nucleic acid molecule, the cartridge including a reaction chamber having a derivatized bottom surface, at least one reagent reservoir, and a pump, in which the reaction chamber, the reagent reservoir, and the pump are fluidically connected to each other. The cartridge uses microfluidic components to link on-board reagent reservoirs via computer controlled valves and plumbing to a reaction chamber having a derivatized bottom surface. In certain embodiments, the cartridge is laminated.

The cartridge is capable of interacting with a channel forming device and a reaction chamber cap. The cartridge, includes a reaction chamber. The reaction chamber has a bottom surface that is composed of a derivatized material. The derivatized bottom surface assists in elongating and fixing nucleic acid molecules, e.g., DNA or RNA, onto the bottom surface of the reaction chamber so that the nucleic acid molecules remain accessible for enzymatic reactions. The nucleic acid molecules are held in an elongated and fixed configuration due to the electrostatic attraction between the nucleic acid molecules and the derivatized surface. The derivatized surface can be composed of any material that can interact with nucleic acid molecules and hold these molecules in an elongated and fixed orientation so that the nucleic acid molecules remain accessible for enzymatic reactions, i.e., produces the electrostatic attraction between the surface and the nucleic acid molecules. Exemplary derivatized materials are shown in U.S. Pat. No. 5,405,519, U.S. Pat. No. 5,599,664, U.S. Pat. No. 6,150,089, U.S. Pat. No. 6,147,198, U.S. Pat. No. 5,720,928, U.S. Pat. No. 6,174,671, U.S. Pat. No. 6,294,136, U.S. Pat. No. 6,340,567, U.S. Pat. No. 6,448,012, U.S. Pat. No. 6,509,158, U.S. Pat. No. 6,610,256, U.S. Pat. No. 6,713,263, and Reslewic et al., Appl Environ Microbiol. 2005 September; 71 (9):5511-22.

In certain embodiments, the derivatized bottom surface is derivatized glass, such as a polylysine-treated glass surface. Methods of derivitizing a glass surface are shown in U.S. Pat. No. 5,405,519, U.S. Pat. No. 5,599,664, U.S. Pat. No. 6,150,089, U.S. Pat. No. 6,147,198, U.S. Pat. No. 5,720,928, U.S. Pat. No. 6,174,671, U.S. Pat. No. 6,294,136, U.S. Pat. No. 6,340,567, U.S. Pat. No. 6,448,012, U.S. Pat. No. 6,509,158, U.S. Pat. No. 6,610,256, U.S. Pat. No. 6,713,263, and Reslewic et al., Appl Environ Microbiol. 2005 September; 71 (9):5511-22.

The cartridge further includes at least one reservoir, and each reservoir further includes a loading port. The reservoir holds reagents that flow to the reaction chamber to interact with the nucleic acids in the reaction chamber. In certain embodiments, the cartridge has four reservoirs, and the cartridge is configured to perform reactions to generate restriction digests of the one or more nucleic acid molecules in the reaction chamber. A first reservoir holds a TE wash reagent, a second reservoir holds a buffer, a third reservoir holds an enzyme, and a fourth reservoir holds a nucleic acid stain. Because each reservoir includes a computer controlled valve, flow of the reagents from each reservoir to the reaction chamber can be controlled.

The cartridge further includes a pump. The pump controls reagent exchange in the reaction chamber, i.e., brings fluids from the reservoirs to the reaction chamber, and also aspirates fluids from the reaction chamber to a reagent waste pad. Because the pump includes aspiration capability, a reagent can be completely removed from the reaction chamber before another reagent is introduced into the reaction chamber, thus avoiding uncontrolled mixing and/or dilution of one reagent by another reagent.

The cartridge components are fluidically connected to each other by methods known to one of skill in the art. The cartridge is composed of multiple plastic polymer layers, for example polycarbonate and polyurethane. A laser is used to burn slots in each layer. When the layers are assembled together, flow channels within the cartridge are formed. The layers are held together with an adhesive and the cartridge is then laminated. The laser is also used to form the reservoirs in the layers. Because the polyurethane layer of the cartridge is flexible, it reacts to pressure. Thus application of pressure or a vacuum results in the polyurethane layer either delivering reagents to the reaction chamber or aspirating reagents from the reaction chamber, i.e., the polyurethane layer acts as the pump for the cartridge. Other similar plastic polymers or materials that are flexible and can react to pressure can also be used in the cartridge instead of polyurethane.

Cartridges of the invention may be used for automated generation of restriction digests of one or more nucleic acid molecules, which can be used to generate an optical map of the one or more nucleic acid molecules. In an exemplary protocol, the channel forming device is loaded with nucleic acid molecules (e.g., DNA or RNA). The channel forming device is configured to mate with the reaction chamber on the cartridge, and interact with the bottom surface (the mapping surface) of the reaction chamber in order to elongate and fix one or more nucleic acid molecules onto the bottom surface of the reaction chamber. Methods of applying nucleic acid molecules to surfaces and elongating and fixing nucleic acid molecules to a surface are shown in U.S. Pat. No. 5,405,519, U.S. Pat. No. 5,599,664, U.S. Pat. No. 6,150,089, U.S. Pat. No. 6,147,198, U.S. Pat. No. 5,720,928, U.S. Pat. No. 6,174,671, U.S. Pat. No. 6,294,136, U.S. Pat. No. 6,340,567, U.S. Pat. No. 6,448,012, U.S. Pat. No. 6,509,158, U.S. Pat. No. 6,610,256, U.S. Pat. No. 6,713,263, and Reslewic et al., Appl Environ Microbiol. 2005 September; 71 (9):5511-22.

Once the nucleic acid molecules have been deposited onto the bottom surface of the reaction chamber, the channel forming device is removed from the reaction chamber, and the reaction chamber cap is placed on top of the reaction chamber, thereby sealing the reaction chamber. The sealed reaction chamber can be of any size, for example having a total internal volume ranging from about 10 μl to about 5 ml depending on the reaction one intends to perform. In a particular embodiment, the sealed reaction chamber has a total internal volume of about 200 μl.

The cartridge is then placed onto a computerized preparation station, which is a computer controlled device having multiple syringe-style air pumps to push fluids through the cartridge in a controlled manner, i.e., programmed volumes and flow rates. An exemplary computerized preparation station is commercially available from Micronics Inc. (Redmond, Wash.). The computerized preparation station also includes a vacuum pressure source for computerized control of the valves connected to each of the reservoirs on the cartridge. The vacuum pressure source also controls the pump in the cartridge. Because the polyurethane layer of the cartridge is flexible, it reacts to pressure. Thus application of pressure or a vacuum results in the polyurethane layer either delivering reagents to the reaction chamber or aspirating reagents from the reaction chamber.

The computerized preparation station also includes a heater/cooler assembly located beneath the derivatized bottom surface of the reaction chamber on the cartridge. An exemplary heating/cooling device is a Peltier device (commercially available from Custom Thermoelectric, Bishopville, Md.). Peltier devices, also known as thermoelectric (TE) modules, are small solid-state devices that function as heat pumps. Generally, the device is formed by two ceramic plates with an array of small Bismuth Telluride cubes in between. Application of a DC current moves heat away from the bottom surface of the reaction chamber or introduces heat to the bottom surface of the reaction chamber. To increase the efficiency of the Peltier module, a thermal interface material can be placed between the Peltier module and the surface. Exemplary thermal interface materials include silicone-based greases (e.g., zinc oxide silicone), elastomeric pads, thermally conductive tapes, and thermally conductive adhesives. In certain embodiments, the heater/cooling device can be a component of the cartridge instead of a component of the preparation station. The heater/cooling device is operated by the preparation station and controls the temperature of the reaction chamber, thus regulating enzymatic digestion of the nucleic acid molecules in the reaction chamber.

Once the cartridge is placed on the preparation station, reagents are loaded into the cartridge, using the loading ports associated with each reservoir. Loading can be accomplished by using any commercially available pipette. Once the reagents have been loaded, the orientation of the cartridge is adjusted to optimize flow of reagents within the cartridge. The cartridge can be placed flat (0° angle) on the surface of the preparation station. Alternatively, the cartridge can be oriented 90° to the surface of the preparation station. Generally, the cartridge can be oriented from about a 0° angle to about a 180° angle with respect to the surface of the preparation station. In a particular embodiment, the cartridge is tilted to a 60° angle with respect the surface of the preparation station in order to optimize reagent flow within the cartridge.

Once the cartridge has been oriented at the optimally determined angle for reagent flow, the preparation station activates the pump in the cartridge and reagents are moved to the reaction chamber from the reservoirs and then aspirated from the reaction chamber to the reagent waste pad. The preparation station controls reagents exchange in the reaction chamber, flow rates, and temperature of the reaction chamber as required to complete washing, enzymatic digestion, and staining of the nucleic acid molecules for generation of restriction digests of the nucleic acid molecules. Further, flow is controlled (e.g., slow flow rates and controlled volumes) such that the nucleic acid molecules are not dislodged from the bottom surface of the reaction chamber.

Once the automated process is completed, the loading ports and any vent holes in the cartridge are sealed e.g., with adhesive tape or labels, and the cartridge is ready for readout on a imaging device, such as a fluorescing microscope/TV/computer system, thereby identifying and/or measuring a single molecule restriction map of the nucleic acid molecule. The restriction map is then used to generate an optical map of the nucleic acid molecule, which can be used, for example for identification of an unknown organism or determining antibiotic resistance of an organism.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

1. A cartridge for characterizing a nucleic acid molecule, the cartridge comprising:
a reaction chamber having a derivatized bottom surface;
at least one reagent reservoir; and
a pump;
wherein the reaction chamber, the reagent reservoir, and the pump are fluidically connected to each other.

2. The cartridge according to claim 1, wherein the cartridge is operably linked to a computerized preparation station.

3. The cartridge according to claim 1, wherein the at least one reagent reservoir is a plurality of reservoirs.

4. The cartridge according to claim 3, wherein each reservoir further comprises a loading port and a computer controlled valve for controlling flow of reagents from the reservoirs to the reaction chamber.

5. The cartridge according to claim 4, wherein a first reservoir holds a TE wash reagent, a second reservoir holds a buffer, a third reservoir holds an enzyme, and a fourth reservoir holds a nucleic acid stain.

6. The cartridge according to claim 1, further comprising a reagent waste pad.

7. The cartridge according to claim 1, further comprising a channel forming device configured to mate with the reaction chamber.

8. The cartridge according to claim 1, further comprising a reaction chamber cap.

9. The cartridge according to claim 1, further comprising a heater/cooling device.

10. The cartridge according to claim 9, wherein the heater/cooling device is located beneath the reaction chamber.

11. The cartridge according to claim 1, wherein the bottom surface of the reaction chamber is composed of derivatized glass.

12. A method for characterizing a nucleic acid molecule, the method comprising:
applying one or more nucleic acid molecules to a derivatized bottom surface of a reaction chamber, wherein the nucleic acid molecules are elongated and fixed onto the surface so that the nucleic acid molecules remain accessible for enzymatic reactions;
flowing reagents to and from the reaction chamber to wash, enzymatically digest, and stain the nucleic acid molecules to obtain one or more restriction digests of the molecule; and
imaging the restriction digests, thereby characterizing the nucleic acid molecule.

13. The method according to claim 12, further comprising constructing an optical map from the restriction digests.

14. The method according to claim 12, wherein prior to said flowing step, the method further comprises applying a cap to the reaction chamber.

15. The method according to claim 12, wherein prior to said flowing step, the method further comprises tilting the cartridge at a 60° angle.

16. The method according to claim 12, wherein flowing is controlled by pumps and valves that are operably connected to a computerized preparation station.

17. The method according to claim 12, wherein the derivatized bottom surface of the reaction chamber is composed of derivatized glass.

18. The method according to claim 12, wherein the nucleic acid molecule is deoxyribonucleic acid or ribonucleic acid.

19. A cartridge for characterizing a nucleic acid molecule, the cartridge comprising:
a reaction chamber having a derivatized bottom surface, wherein a nucleic acid molecule is elongated and fixed onto the derivatized bottom surface of the reaction chamber so that the nucleic acid molecule remains accessible for enzymatic reactions;
at least one reagent reservoir; and
a pump;
wherein the reaction chamber, the reagent reservoir, and the pump are fluidically connected to each other.

20. A system for characterizing a nucleic acid molecule, the system comprising:
a cartridge comprising: a reaction chamber having a derivatized bottom surface, wherein a nucleic acid molecule is elongated and fixed onto the derivatized bottom surface of the reaction chamber so that the nucleic acid molecule remains accessible for enzymatic reactions; at least one reagent reservoir; and a pump; wherein the reaction chamber, the reagent reservoir, and the pump are fluidically connected to each other; and
a computerized preparation station operably linked to the cartridge, wherein the station controls flow of reagents to and from the reaction and chamber.
